(19) United States
(12) Patent Application Publication
(10) Pub. No.: US 2014/0324034 A1
(43) Pub. Date: Oct. 30, 2014

(54) SYSTEMS AND METHODS FOR FACILITATING ROBOTIC SURGICAL LASER PROCEDURES

(71) Applicant: LUMENIS LTD, Yokneam litI (IL)

(72) Inventors: Gelstein Assaf, Haifa (IL); Nissim Hai, Netanya (IL)

(21) Appl. No.: 14/357,036

(22) PCT Filed: Nov. 9, 2012
(86) PCT No.: PCT/IB2012/056294

§ 371 Date: May 8, 2014

Related U.S. Application Data
(60) Provisional application No. 61/558,521, filed on Nov. 11, 2011.
(60) Provisional application No. 61/567,840, filed on Dec. 7, 2011.

(51) Int. Cl. A61B 18/22; A61B 19/00

(52) U.S. Cl. CPC A61B 18/22; A61B 19/2203; A61B 2017/00464
USPC 606/10

(57) ABSTRACT

A system of surgical accessories for a surgical robotic arm is provided, the system comprising: at least one working channel coupled to an optical fiber; a universal accessory adapter having: a first end coupled to a working end of the surgical robotic arm, a second end configured with an opening that exposes the at least one working channel; and an accessory fastener, wherein said universal accessory adapter is configured for securing an accessory device to the second end.

### BACKGROUND

Surgical robots are quickly gaining acceptance for performing surgical procedures on human patients. When controlled by skilled physicians, these robots can often provide a platform for delivering surgical treatments with a degree of precision greater than the physician could provide using traditional surgical methods alone. Robotic arms on these surgical robots today facilitate many traditional surgical instruments such as scissors, hooks, spatula, forceps, scalpel blades and graspers.

Some robotic arms are also equipped with laser instruments. For example disclosed in U.S. Pat. No. 6,714,841 is the use of laser for marking in remote robotic laparoscopic surgeries. Disclosed in US 2010204713 are similar procedures for distance measurements. In U.S. Pat. No. 8,257,303 a robotic procedure with flexible endoscope for intravascular applications is taught. US2009248041 discloses both laser marking and cutting in robotic surgical systems. US200924804141 teaches, for example, the use of the generic Intuitive robotic arm wrist joint design with an optical fiber. However, key features which are specifically related to laser technology and its performances such as fiber mechanical flexibility etc, are not addressed by the prior art systems.

For the reasons stated above and for other reasons stated below which will become apparent to those skilled in the art upon reading and understanding the specification, there is a need in the art for a system for facilitating robotic surgical laser procedures.

### SUMMARY

The present invention provides a system for facilitating robotic surgical laser procedures and will be understood by reading and studying the following specification.

In a first aspect of the invention, there is provided a system of surgical accessories for a surgical robotic arm, the system comprising: at least one working channel coupled to an optical fiber; a universal accessory adapter having: a first end coupled to a working end of the surgical robotic arm, a second end configured with an opening that exposes the at least one working channel; and an accessory fastener, wherein said universal accessory adapter is configured for securing an accessory device to the second end.

### BRIEF DESCRIPTION OF THE DRAWINGS

Embodiments of the present invention can be more easily understood and further advantages and uses thereof more readily apparent, when considered in view of the description of the preferred embodiments and the following figures in which:

FIGS. 1a and 1b are diagrams illustrating a robotic arm including a range restriction device.

FIG. 2 is a diagram of a universal accessory adapter.

FIGS. 3a and 3b are diagrams illustrating a waveguide tip accessory coupled to a universal accessory adapter.

FIG. 4 is a diagram illustrating an embodiment of a bladed tip accessory.

FIG. 5 is a diagram illustrating a spatula accessory according to one embodiment of the present invention.

FIGS. 6 and 7 are diagrams illustrating backstop tip accessories.

FIGS. 8a and 8b illustrate two embodiments of accessories coupled with an integrated tip universal accessory adapter.

FIGS. 9 and 10 are diagrams illustrating an offset entry accessory of one embodiment of the present invention.

FIG. 11 is a diagram illustrating an embodiment of an offset fiber delivery assembly, according to the present invention.

FIG. 12 is a diagram illustrating a robotic arm including an alternate range restriction device according to one embodiment of the present invention.

FIG. 13 is a diagram illustrating a system of one embodiment of the present invention.

FIGS. 14 and 15 are diagrams illustrating a Trocar introducer according to one embodiment of the present invention.

FIGS. 16 and 17 are embodiments of a sealing member.

FIGS. 18a and 18b are diagrams illustrating embodiments of a collar on an off-set introducer.

FIG. 19 is a diagram illustrating the grasping of the optical fiber tip by the robotic arm, via the collar of the embodiment illustrated in FIG. 18b.

FIGS. 19, 20 and 21 are diagrams illustrating further embodiments of the collar of an off-set introducer.

In accordance with common practice, the various described features are not drawn to scale but are drawn to emphasize features relevant to the present invention. Reference characters denote like elements throughout figures and text.

### DETAILED DESCRIPTION

In the following detailed description, reference is made to the accompanying drawings that form a part hereof, and in which is shown by way of specific illustrative embodiments in which the present invention may be practiced. These embodiments are described in sufficient detail to enable those skilled in the art to practice the invention, and it is to be understood that other embodiments may be utilized and that logical, mechanical and electrical changes may be made without departing from the scope of the present invention. The following detailed description is, therefore, not to be taken in a limiting sense.

The following are definitions of terms used in the description and claims.

“Working channel” is a conduit in the robotic arm which allows the insertion of various instruments through the robotic arm.

“Universal accessory adapter” is an accessory which allows the attachment of the various instruments to the robotic arm.

“Accessory fastener” is an attachment means allowing for the attachment of the various instruments to the robotic arm.

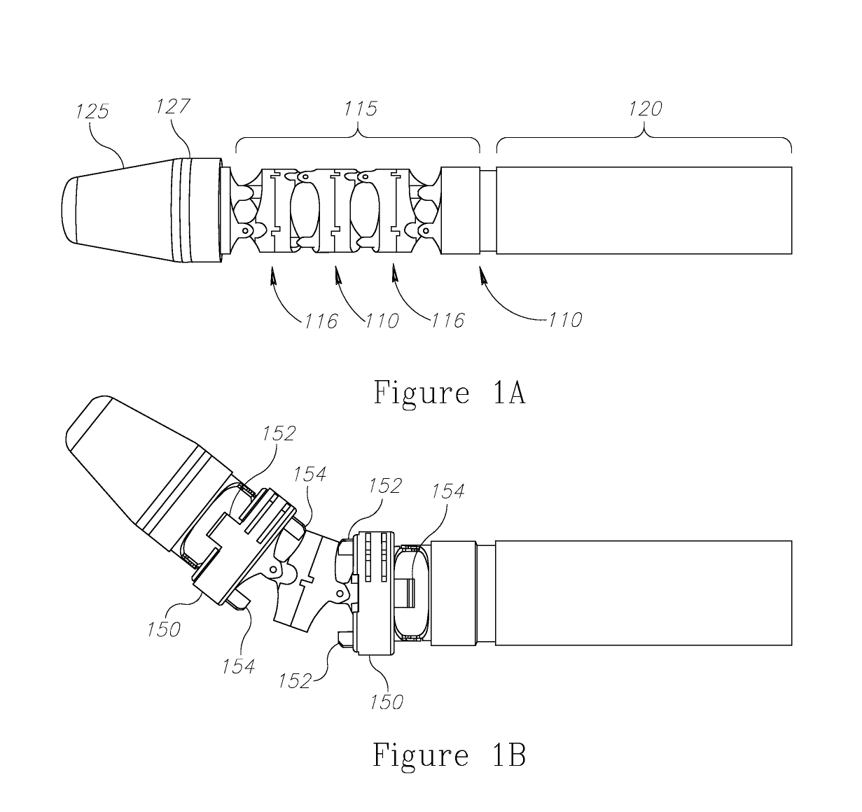

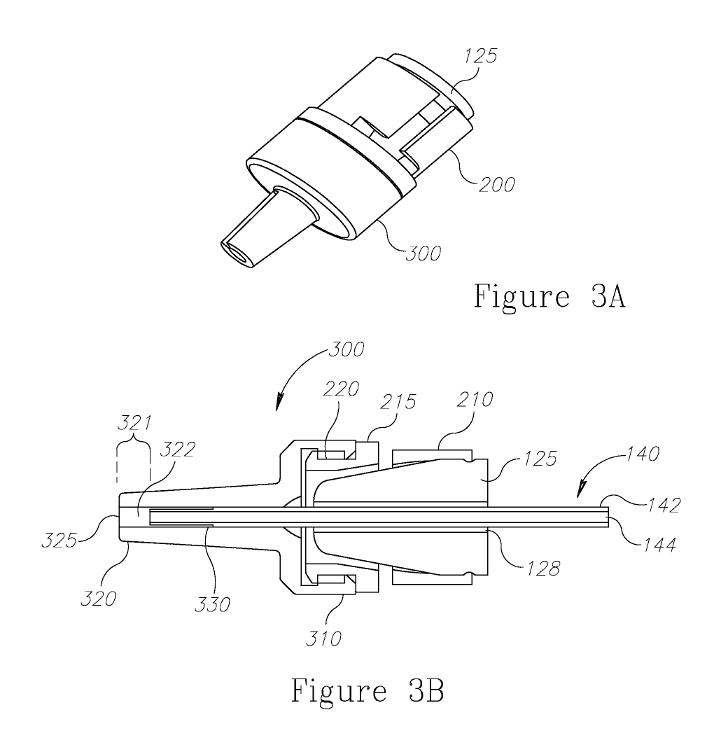

FIG. 1 illustrates one embodiment of a range restriction device for use on a robotic arm 110 configured for use for laser surgical procedures. The robotic arm 110 comprises a sheath 125 coupled to an arm 120 via a wrist assembly 115. The wrist assembly 115 comprises a series of hinged links 116 which allow movement with multiple degrees of freedom. In this configuration, the robotic arm 110 comprises what is commonly referred to in the surgical robotics industry as an Introducer. The robotic arm 110 includes at least one working channel, or conduit 128 (shown in FIG. 3b) for introducing instruments used during surgical procedures. For example, in one embodiment, the robotic arm 110 comprises a 5 Fr. Introducer for the Intuitive da Vinci family of surgical robots configured to deliver an optical fiber instrument.

The wrist assembly 115 allows the robotic arm 110 to move with multiple degrees of freedom for maneuvering and positioning. Whilst this range of movement is advantageous for many surgical procedures, when the working channel 128 is provided with a laser fiber, the range of motion of the wrist assembly 115 needs to be restricted in order not to bend the laser fiber more than the laser fiber's rated bend radius permits. If the laser fiber's bend radius limit is exceeded, then the laser beam may perforate the cladding material and light may leak from the fiber and risk the patient and/or the fiber may break. In one embodiment, to restrict the range of motion available, one or more range restriction devices 150 are affixed to the wrist assembly 115. In the embodiment shown in Figure lb, the range restriction devices 150 comprise ring clamps, each having a first axis limiter 152 and a second axis limiter 154. When the wrist assembly 115 moves about a particular hinged link, the limiters 152 and 154 provide physical stops that restrict the range of motion of that particular hinged link. The movement about that link can be either partially limited or completely restricted by the limiters 152, 154. By limiting the movement at selected hinged links 116, the range restriction devices 150 modify the range of movement available to the wrist assembly 115 so that excessive bending and/or twisting of the laser fiber does not occur.

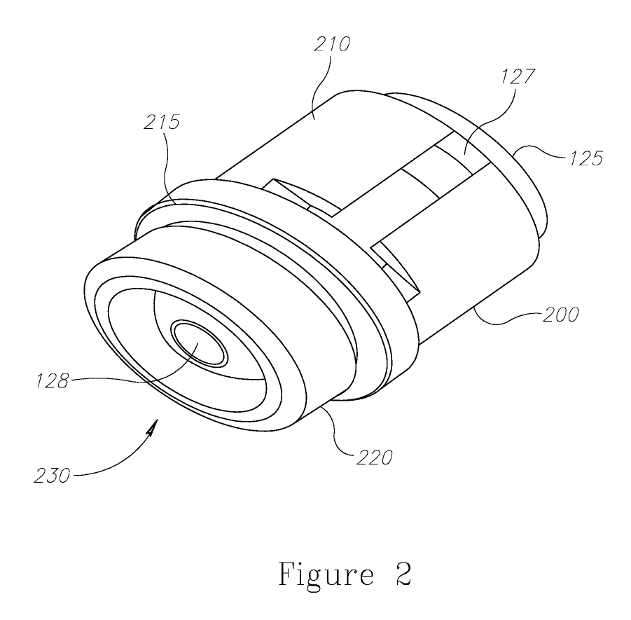

FIG. 2 illustrates a universal accessory adapter 200, which includes a snap ring member 210 that engages a groove 127 located around the periphery of sheath 125. In other embodiments, other fastening means are used to secure universal accessory adapter 200 to the robotic arm 110. The universal accessory adapter 200 further includes an accessory fastener 220 that includes an opening 230 exposing the working channel 128 of robotic arm 110. The accessory fastener 220 provides an attachment point to secure one of many possible accessories (described later in this specification) to the universal accessory adapter 200 and thus also to robotic arm 100. In the embodiment illustrated in FIG. 2, the accessory fastener 220 is profiled as a ring around the circumference of adapter 200 that facilitates snap-on attachment of the accessories. In one such embodiment, the accessory fastener 220 includes a stop feature 215 to help further position the accessory. In other embodiments, the accessory fastener 220 provides a threaded profile for screw-on fastening of accessories.

FIGS. 3a and 3b are diagrams illustrating a waveguide tip 300 accessory according to one embodiment of the present invention. The waveguide tip 300 comprises a fastening member 310 configured to engage with the accessory fastener 220 of the universal accessory adapter 200. A laser fiber 140 is delivered through the working channel 128 via sheath 125, into tip member 320 of waveguide tip 300. Optical energy emitted from laser fiber 140 follows the channel 322 of the tip member 320, exiting at opening 325 for delivery to a patient tissue.

In order to prevent optical fiber 140 from extending through the opening 325, the channel 322 is provided with a fiber locking means 330. In one embodiment of the present invention, at fiber locking means 330 the diameter of the channel 322 becomes smaller than that of the optical fiber 140. For example, as illustrated in FIG. 3b, the optical fiber 140 includes a fiber core 144 surrounded by a cladding 142. Prior to the fiber locking means 330, channel 322 has a sufficient diameter to accommodate both fiber core 144 and cladding 142. At the fiber locking means 330, the diameter of the channel 322 becomes smaller so that only the fiber core 144 can pass through. By stripping the cladding 142 from a length (for example, 3 mm) off the end of the optical fiber 140, the fiber locking means 330 will only permit that stripped portion of the optical fiber 140 to proceed toward opening 320. In one embodiment the diameter change is instant by a step-like geometry. In yet another embodiment, the diameter change is gradual by having, for example, a tapered channel 322. As Illustrated in FIG. 3b, the position of the fiber locking means 330 and the length of cladding 142 stripped from optical fiber 140 are coordinated so that there remains a gap 321 between the end of the fiber 140 and the opening 325. In the embodiment of the fiber locking means with a gradual diameter change of the channel 330, a range of fiber diameters can be stopped once reaching a size matching between the fiber 140 outer diameter and internal channel 330 diameter. A gradual diameter change of the channel 330 enables a dynamic range of fiber diameters which can be stopped by the same waveguide tip 300 and its locking feature 330. In this embodiment the channel 322 has to be long enough to accommodate this dynamic range of external fiber 140 diameters while still having gap 321 unoccupied by the fiber tip. Maintaining the gap 321 ensures that there is no possibility that fiber 140 is damaged by coming in direct contact with the patient's tissues. Because many varieties of optical fiber 140 are available with differing core 144 diameters and cladding 142 thicknesses, a corresponding variety of different implementations of waveguide tip 300 are contemplated as within the scope of the embodiments of the present invention.

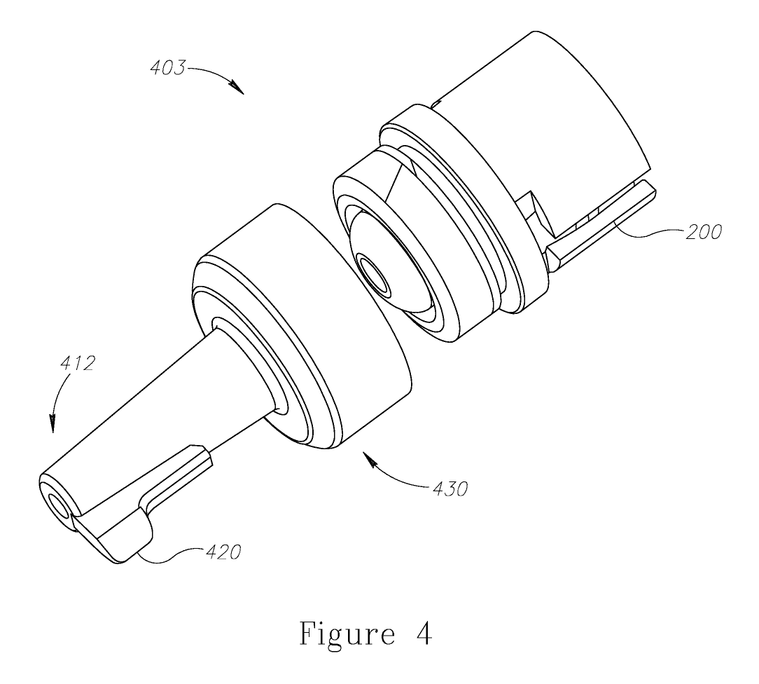

FIG. 4 is a diagram illustrating a bladed tip accessory 430. Shown generally at 403, a bladed tip accessory 430 is coupled to a universal accessory adapter 200. Bladed tip accessory 430 is identical to the waveguide tip 300 with the exception that the tip member 412 now includes a curved blade element 420. Curved blade element 420 provides a surgeon with a means for performing minor physical manipulation and/or incision of tissues using robotic arm 110. As would be appreciated by one of ordinary skill in the art reading this disclosure, in still other embodiments, other blade shapes can be utilized. Also shown generally at 403 is an exploded view illustrating how the bladed tip accessory 330 engages with the universal accessory adapter 200.

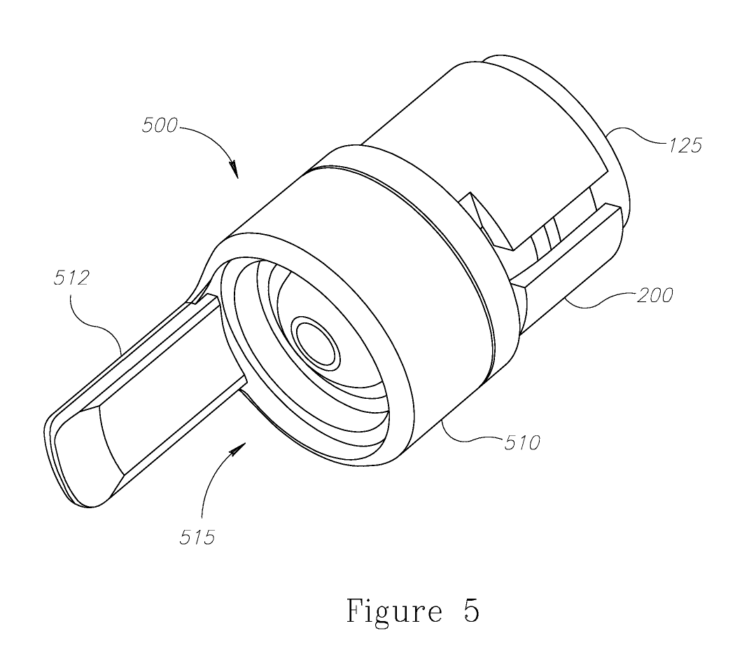

FIG. 5 is a diagram illustrating a spatula 500 accessory coupled to the universal accessory adapter 200. The spatula 500 comprises an attachment ring 510 which further includes an opening 515 that exposes the working channel 128. Coupled to the attachment ring 510 is at least one spatula tip 512. In the embodiment shown in FIG. 5, the spatula tip 12 extends from the attachment ring 510 in a direction aligned parallel with the optical path of light exiting working channel 128. In other embodiments, spatula tip 512 will be angled off - parallel with respect to said optical path. Spatula tip 512 provides at least two functions. First, it functions as a physical limiter, preventing working channel 128 from coming any closer to the tissue under treatment than the length of spatula tip 512 will permit. As such, different implementations will encompass spatula tips of different lengths. Second, it functions as a means for performing minor physical manipulation of tissues under treatment.

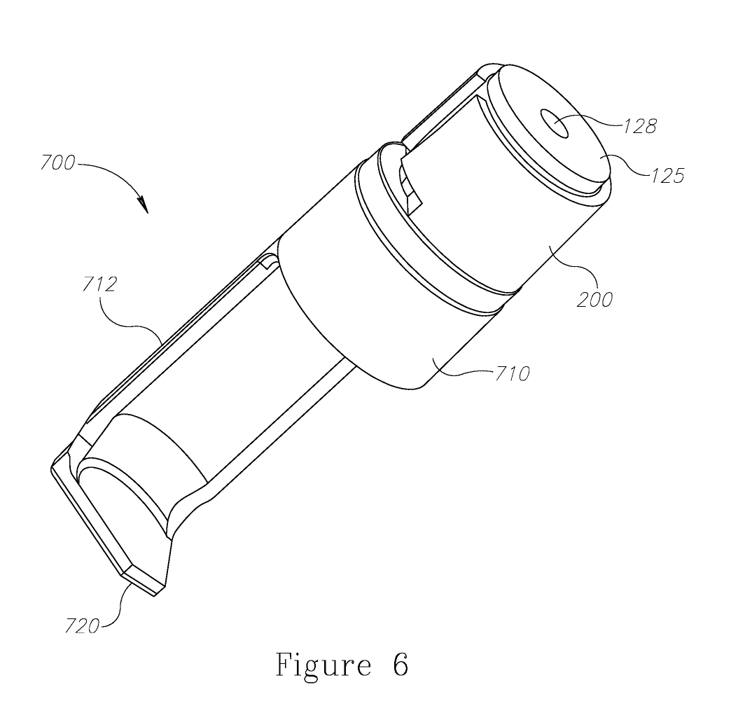

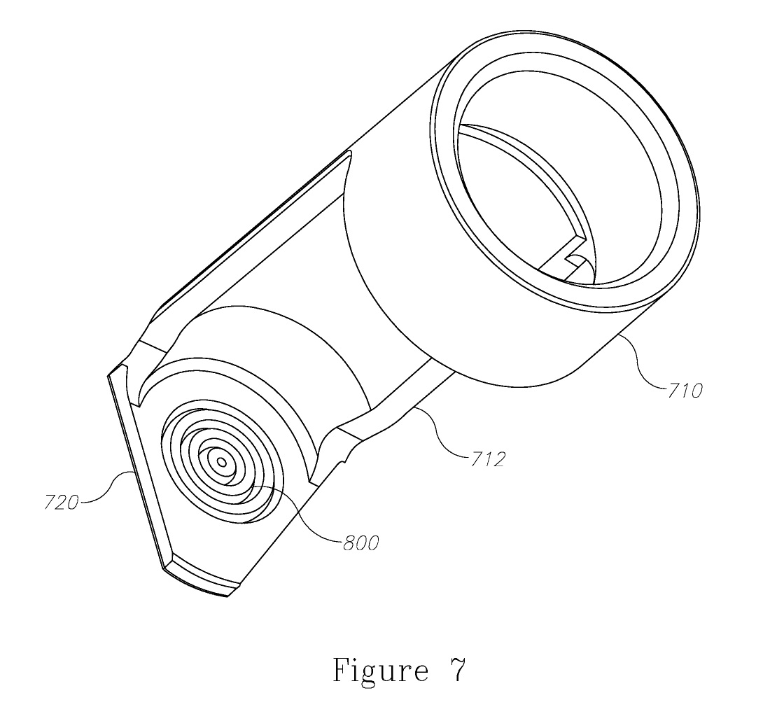

FIGS. 6 and 7 are diagrams illustrating an embodiment of the backstop tip accessory 700. A backstop tip accessory is useful for treating a target tissue when you want to protect tissues behind the target tissue. For some applications, a backstop tip accessory further functions as a heat sink that absorbs excess thermal energy generated by the laser light in order to reduce collateral damage.

FIG. 6 illustrates a first backstop tip accessory 700 of one embodiment of the present invention coupled to a universal accessory adapter 200. Backstop tip accessory 700 comprises an attachment ring 710 having an opening that the exposes working channel 128. A backstop 720 is coupled to the attachment ring 710 via extension 712. In the embodiment shown in FIG. 6, backstop 720 is positioned in a plane normal to the optical path of light exiting working channel 128. In other embodiments, the plane of backstop 720 may be oriented into other positions that are otherwise angled with respect to the optical path of light exiting working channel 128. In operation, in one embodiment, a tissue under treatment is placed within the treatment area between the backstop 720 and attachment ring 710. Laser energy emitted from the working channel 128 enters treatment area through an opening. Any energy traversing through the tissue to reach backstop 720 is blocked by backstop 720 from further penetrating into other tissues.

FIG. 7 illustrates a backstop tip accessory 700 of one embodiment of the present invention coupled to the universal accessory adapter 200. The backstop tip accessory 700 comprises a backstop 720 which is coupled to the attachment ring 710 via an extension 712. The backstop 720 has a wedge shape. In other embodiments, the backstop of a backstop tip accessory will include still other shapes. In alternate implementations, backstop tip accessories are contemplated as having extensions of various lengths in order to accommodate tissues of different thicknesses.

As illustrated in FIG. 7, in still other embodiment, the surface of the backstop is textured or otherwise patterned. For example, in FIG. 7, the surface of the backstop 720 is shown having a textured pattern 800 of concentric circles. In operation, the textured pattern 800 functions to further disperse laser light reaching backstop 720, thus reducing the energy of any light reaching collateral tissues not intended for treatment.

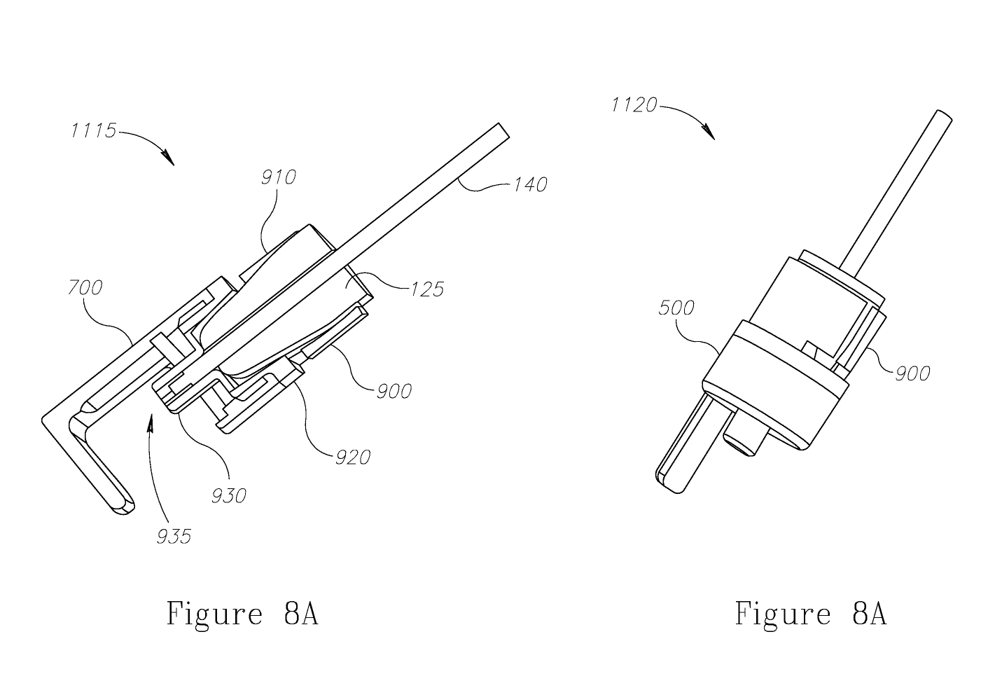

FIGS. 8a and 8b are diagrams illustrating an integrated tip universal accessory adapter 900. That is, integrated tip universal accessory adapter 900 comprises a combination of the features of universal accessory adapter 200 and a waveguide tip integrated into a single attachment to sheath 125. In the embodiment illustrated in FIG. 8a, the integrated tip universal accessory adapter 900 includes a snap ring member 910 that engages a groove 127 located around the periphery of sheath 125. In another embodiment, other fastening means are used to secure integrated tip universal accessory adapter 900 to sheath 125.

Referring to FIG. 8a, integrated tip universal accessory adapter 900 further comprises an accessory fastener 920 that includes an integrated tip member 930. Accessory fastener 920 is configured to secure accessories to the integrated tip universal accessory adapter 900 and thus also to sheath 125. In the embodiment illustrated in FIG. 8a, accessory fastener 920 is profiled as a ring around the circumference of adapter 900 that permits the accessories to snap on. In other embodiments, accessory fastener 920 provides a threaded profile for fastening accessories. Laser fiber 140 is provided through working channel 128 of sheath 125, extending into tip member 930. Optical energy emitted from the laser fiber 140 follows the channel of the tip member 930, exiting at opening 935.

In order to prevent the optical fiber 140 from extending through opening 935, the channel is provided with a fiber locking feature, which functions in the same fashion as the fiber locking feature 330 described above by reducing the diameter of channel so that the stripped fiber core 144 can continue through the channel towards the opening 935, but not portions of fiber 140 where the cladding 142 remains. The position of the fiber locking feature and the length of cladding 142 stripped from optical fiber 140 are coordinated so that there remains a gap between the end of fiber 140 and opening 935. In this way, there is no possibility that fiber 140 will come in direct contact with a patient's tissues. Because many varieties of optical fiber 140 are available with differing core 144 diameters and cladding 142 thicknesses, a corresponding variety of different integrated tip universal accessory adapter 900 are contemplated as within the scope of embodiments of the present invention. Furthermore, the fiber locking feature may provide a diameter change that is instant by a step-like geometry or a gradual diameter change. A gradual diameter change of the channel enables a dynamic range of fiber diameters in the same manner as discussed above with respect to fiber locking feature 330.

FIGS. 8a and 8b are diagrams illustrating alternate embodiments of an integrated tip universal accessory adapter 900 combined with the accessories illustrated in FIGS. 5 to 7 above. View 1115 (FIG. 8a) provides a cross-sectional view of the combination of an integrated tip universal accessory adapter 900 and the backstop tip accessory 600. A combination of the integrated tip universal accessory adapter 900 with spatula 500 is also illustrated generally at view 1120 (FIG. 8b). In this embodiment, the attachment ring 510 of the spatula 500 fastens to accessory fastener 920 in the same manner as it would fasten to accessory fastener 220 of universal accessory adapter 200 with the integrated tip member 930 protruding through opening 515.

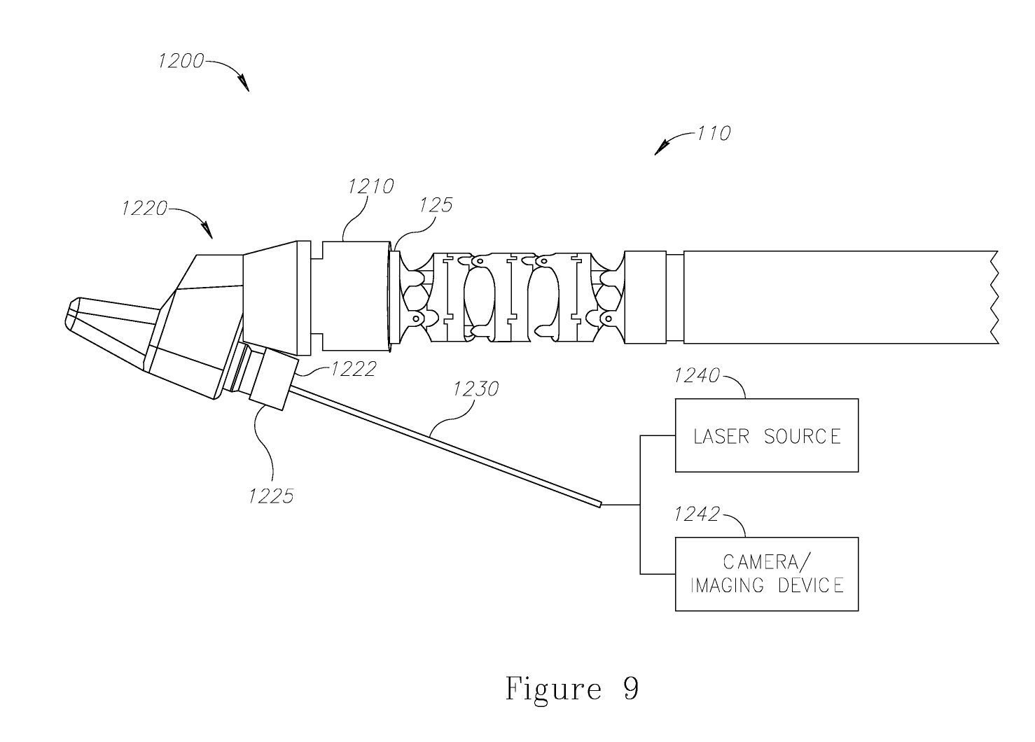

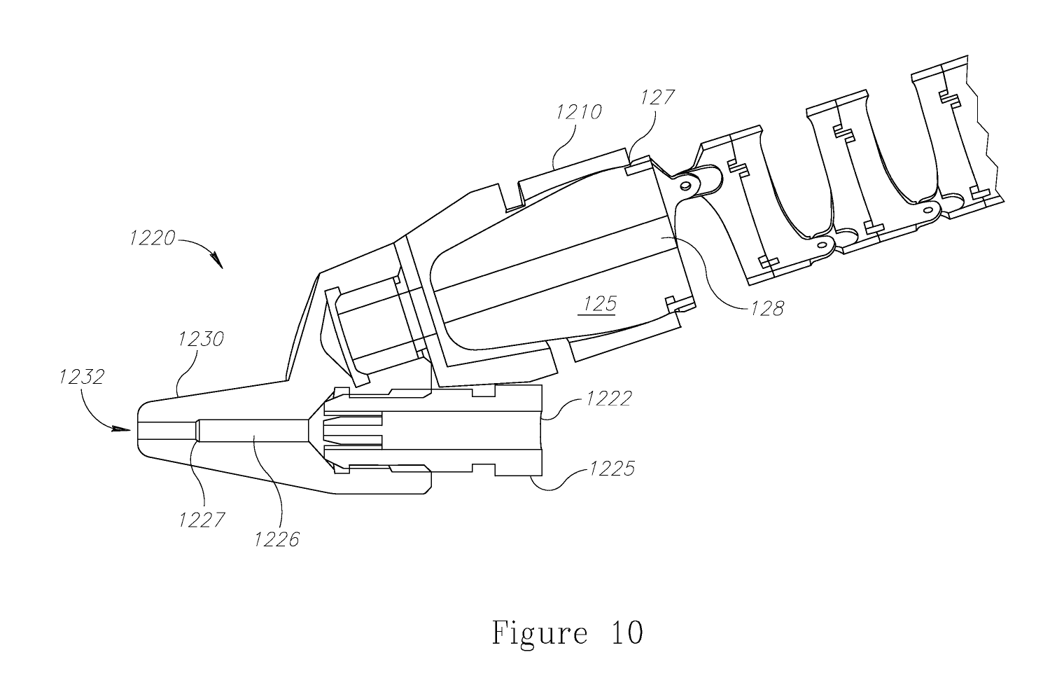

FIGS. 9 and 10 are diagrams illustrating an offset entry accessory 1200 of one embodiment of the present invention. The offset entry accessory 1200 provides a means for adding a second fiber to the robotic arm 110 at a secondary angle from that provided by channel 128. A secondary fiber 1230 is used, for example, to introduce optical energy from a second laser source 1240 at a different angle than that provided by fiber 140 from channel 128 and/or laser light of different working parameters such as a different wavelength, repetition rate (frequency) and/or spot size. Furthermore the optical energy from a second laser source 1240 can be a pulsed laser or a continuous wave laser different than that provided by channel fiber 140. Moreover, the secondary fiber 1230 can alternately be used with an optical fiber camera 1242 for observing performance of the laser treatment by channel 128.

In another embodiment, the offset entry accessory 1200 provides a means for adding a second energy source to robotic arm 110 at a secondary angle for that provided by channel 128. The second energy source can be, for example, a laser, ultrasound, radio frequency, microwave, or a cryogenic tip. In one embodiment, the second energy source targets the same tissue which is targeted by the first fiber 140 provided by channel 128. In another embodiment, the second energy source targets an adjacent tissue to the tissue which is targeted by the first fiber 140 provided by channel 128. Yet in another configuration, the offset entry accessory 1200 is configured to provide multiple offset entries for multiple energy sources, each configured to provide access to a separate energy delivery mechanism.

Offset entry accessory 1200 includes a ring member 1210. In one embodiment, the ring member 1210 is a snap ring member that engages a groove 127 located around the periphery of the sheath 125. Channel 128 provides a means to deliver laser light from a laser fiber (such as fiber 140 described above) to a target tissue. Offset entry accessory 1200 further includes an offset fiber assembly 1220 coupled to ring member 1210. In one embodiment, the ring member 1210 comprises a universal accessory adapter 200 or integrated tip universal accessory adapter 900, wherein offset fiber assembly 1220 is fastened thereto in a manner such as describe above. In other embodiments, the ring member 1210 and offset fiber assembly 1220 are integrated as a single member.

Secondary fiber 1230 enters the offset fiber assembly 1220 at fiber entry 1222. Referring to the cross-section of offset entry accessory 1200 provided by FIG. 10, offset fiber assembly 1220 includes a fiber guide tube holder 1225 which secures and orients the secondary fiber 1230 within the offset fiber assembly 1220. The offset fiber assembly 1220 is aligned with respect to channel 128 so that the optical paths of light exiting from channel 128 and offset fiber assembly 1220 will impinge on a tissue at the same point, but at different angles.

Offset fiber assembly 1220 further includes a tip 1230 having a channel 1226 provided with a fiber locking means 1227, which functions to limit the fiber 1230 from penetrating through opening 1232 as describe above with respect to fiber locking means or features 330 and 940. As discussed above, the position of fiber locking feature 330 is coordinated with and the length of cladding stripped from optical fiber 1230 so that there remains a gap between the end of fiber 1320 and the opening 1232. Again, because many varieties of optical fiber 1320 are available with differing core diameters and cladding thicknesses, a corresponding variety of different implementations of the fiber offset fiber assembly 1220 are contemplated as within the scope of the embodiments of the present invention. Furthermore, the fiber locking feature 1220 may provide a diameter change that is instant by a step-like geometry or a gradual diameter change. A gradual diameter change enables a dynamic range of fiber diameters in the same manner as discussed above with respect to fiber locking feature 330.

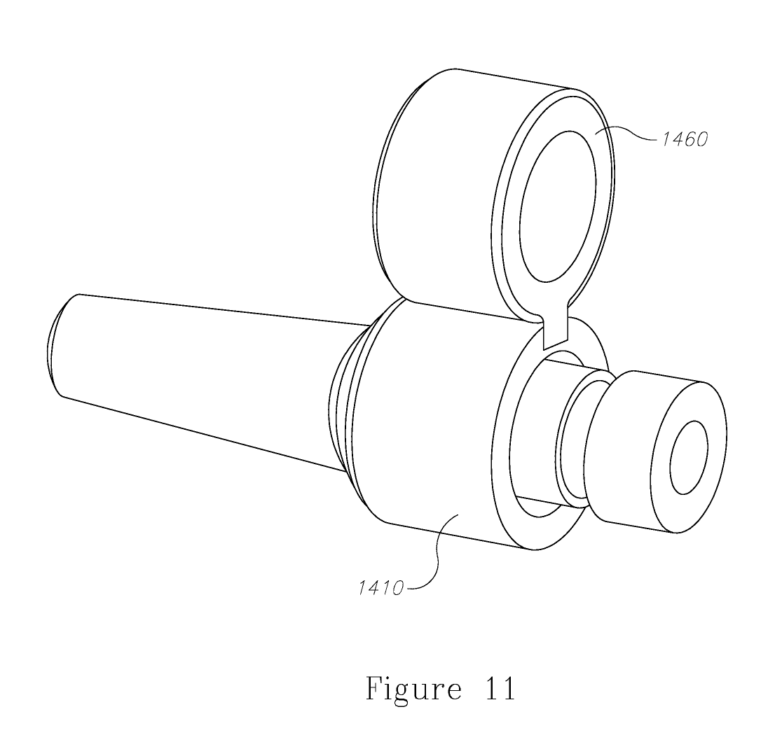

FIG. 11 illustrates another embodiment of the offset fiber delivery assembly 1410 having a ring 1460 for grasping rather than flipper. Both ring 1460 and the flipper provide a handle member for grasping hold of the offset fiber delivery assembly 1410. In one embodiment of the present invention, ring 1460 or a flipper are made of a medical grade polymer or elastomer. In yet another embodiment, the ring 1460 or the flipper are made of a metal. In this embodiment, the metal may be a paramagnetic metal and the grasper may have an integrated magnet element to ease the introduction between the grasper and ring 1460 or flipper. In another embodiment the grasper is made of a paramagnetic metal and ring 1460 or flipper incorporate a magnet source.

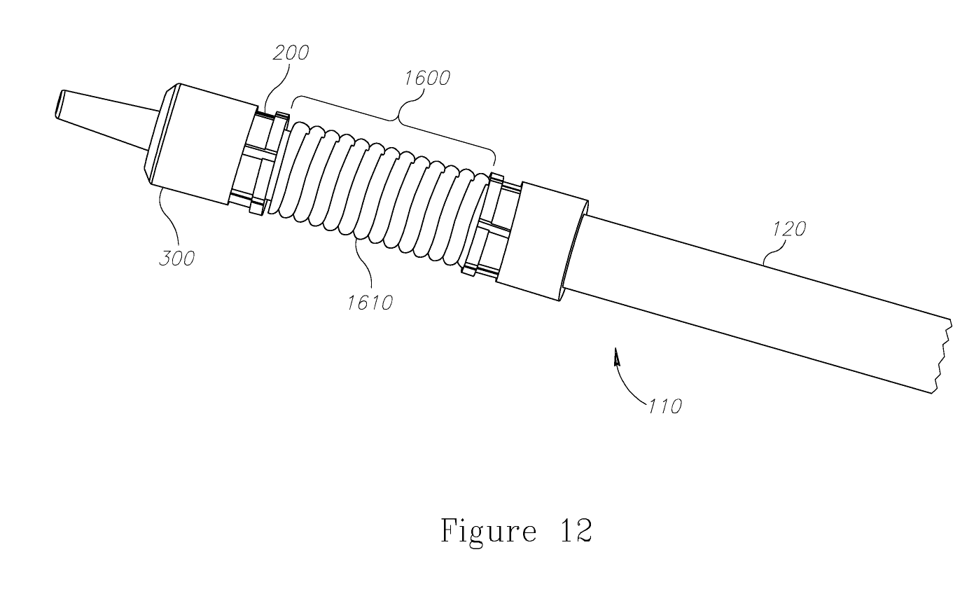

FIG. 12 illustrates a robotic arm 110 with an alternate movement inhibiting device 1600. In FIG. 12, a waveguide tip 300 is coupled to robotic arm 110 via universal accessory adapter 200. As opposed to the range restriction devices 150 comprising rings clamps as shown in Figure lb, movement inhibiting device 1600 comprises a coiled spring assembly 1610 that wraps around the wrist assembly 115 of the robotic arm 110. The tension provided by the coiled spring assembly 1610 prevents the robotic arm 110 from having positions that would bend the laser fiber 140 in channel 128 more than the laser fiber's rated bend radius permits.

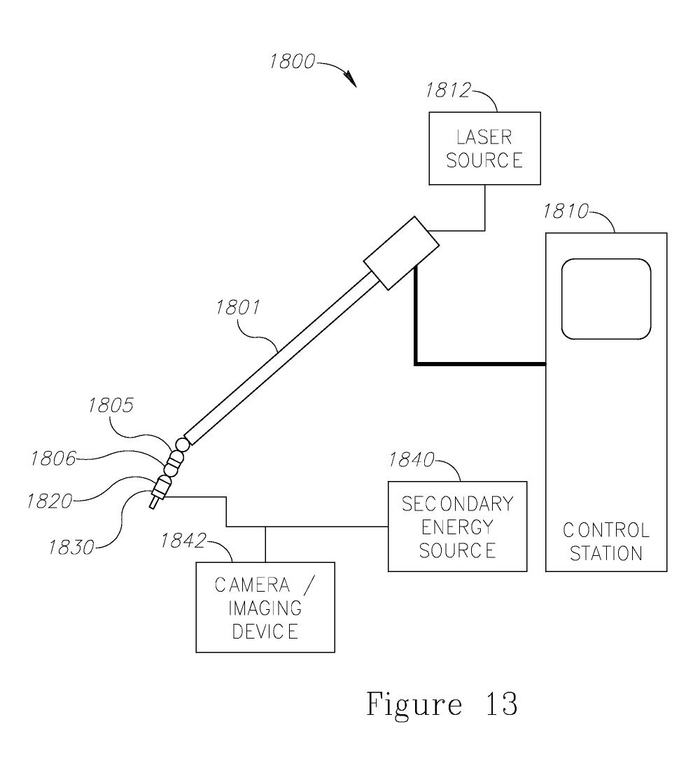

FIG. 13 is a diagram illustrating a surgical system 1800 of one embodiment of the present invention. System 1800 includes a surgical robotic arm 1801 having a plurality of degrees of freedom of movement about wrist assembly 1805. Robotic arm 1801 includes at least one working channel configured with an optical fiber. The robotic arm 110 discussed above having a working channel 128 with optical fiber 140 is one example of an embodiment of robotic arm 1801. Accordingly, in one embodiment, robotic arm 1801 comprises the sheath 125 coupled to the arm 120 via wrist assembly 1805. In one embodiment, the wrist assembly 1805 comprises a series of hinged links such as described above with respect to wrist assembly 115. As such, in alternate embodiments either the above described ring clamps, or a coiled spring assembly may be utilized to at least partially restrict at least one degree of freedom of robotic arm 1801. As shown in FIG. 13, the robotic arm 1801 is further coupled to a control station 1810 that controls the positioning of the robotic arm 1801 and wrist assembly 1805. In one embodiment, a laser energy source 1812 is coupled to optical fiber 140. In other embodiments, laser energy source 1812 is integrated into either control station 1810 or robotic arm 1801.

In one embodiment, fastened to robotic arm 1801 is a universal accessory adapter 1820. A first end of the universal accessory adapter 1820 is coupled to the working end of the surgical robotic arm 1801. The second end of the universal accessory adapter 1820 includes an accessory fastener for attaching accessory device 1830. In alternate embodiments, the universal accessory adapter 1820 comprises either the universal accessory adapter 200 or an integrated tip universal accessory adapter 900 described above. As such, in an alternate embodiment, accessory device 1830 may include any of the accessories used with the universal accessory adapter 200 or the integrated tip universal accessory adapter 900 as described above with respect to any of the figures above.

However, embodiments of the present invention are not limited to just those accessories and in other embodiments, other accessory devices are used. In one embodiment, an offset fiber accessory (such as described with respect to offset entry accessory 1200 in FIGS. 9 and 10) is instead coupled to robotic arm 1801. Further, in one embodiment, a secondary energy source 1840 is coupled to the accessory device 1830 as means for adding another energy source to robotic arm 1801 at a secondary angle. Similarly, an optical imaging device 1842 such as a fiber optic camera can be coupled to the accessory device 1830 for observing or otherwise proving feedback on the work performed via working channel 128 of robotic arm 1801.

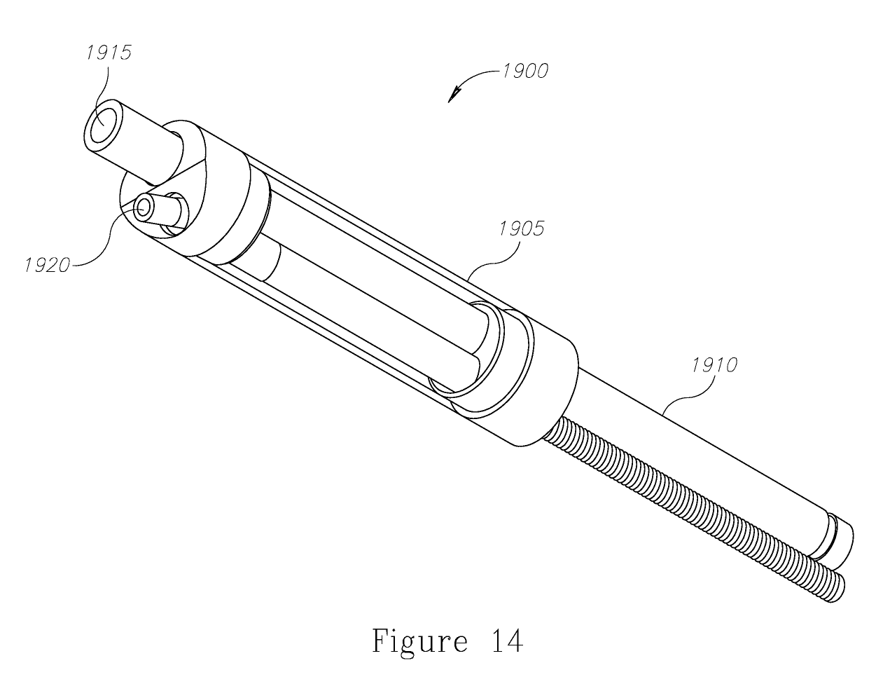

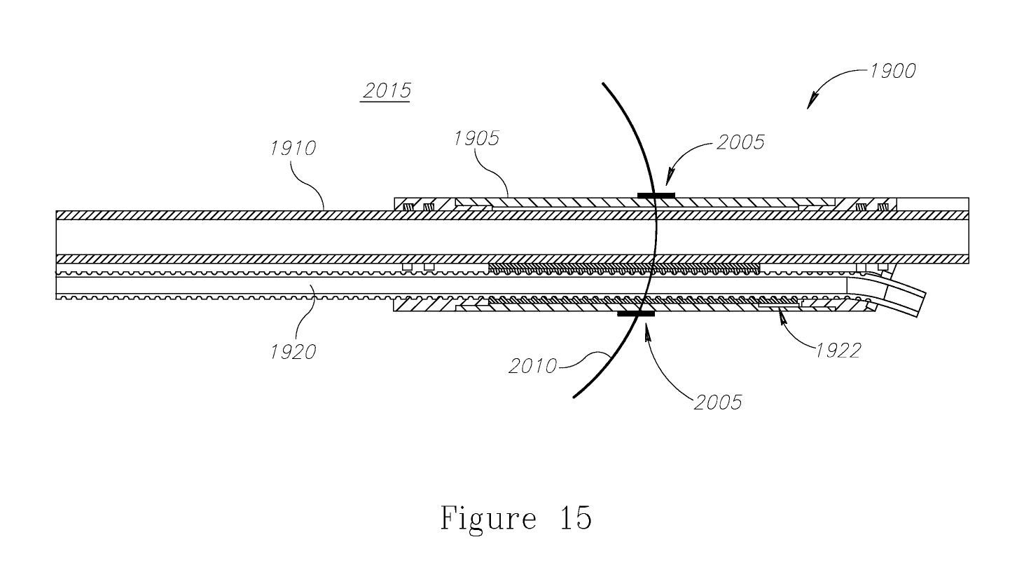

FIGS. 14 and 15 illustrate a Trocar introducer 1900 of one embodiment of the present invention. FIG. 14 illustrates a Trocar introducer 1900 coupled onto a robotic arm 1910 having at least one working channel 1915. In one embodiment, the robotic arm 1910 is a robotic arm such as robotic arm 110 described in any of the figures above. In another embodiment, the robotic arm 1910 comprises other configurations. For example, as illustrated in FIG. 15, in at least one embodiment, the robotic arm 1910 includes a working channel 1915 configured as a needle driver. The Trocar introducer 1900 further includes an external channel 1920 to provide an off-set introducer. Portions of the robotic arm 1910 and external channel 1920 are encased within an outer sleeve 1905 which functions to secure the external channel 1920 to robotic arm 1910. Here only one off-set introducer is shown, however, in the same manner, further off-set introducers may be added to allow delivery of multiple lasers to the working site. The lasers may deliver different wavelengths of energy or different types of energy.

Certain surgical procedures, such as laparoscopic procedures, utilize inflation of the abdomen volume (using inert gases for example) to allow room for viewing, manipulation of internal organs, and manipulation of instruments. One such application of the Trocar introducer 1900 is illustrated by FIG. 15. The Trocar introducer 1900 is shown penetrating a patient's abdominal wall 2010 via Trocar introducer 2005. Because the intra-abdominal space 2015 needs to remain inflated, there must be sealing in and around the Trocar introducer 2005. As such, the internal penetrations through the Trocar introducer 1900 for the robotic arm 1910 and external channel 1920 are also sealed, such as by a silicon ring 1922. In yet another embodiment of an Trocar introducer, that facilitates an offset channel, a molded element (for example, a silicon element) includes two channels—one to accept robotic arm 1910 and a second to accept the external channel 1920 of the offset introducer.

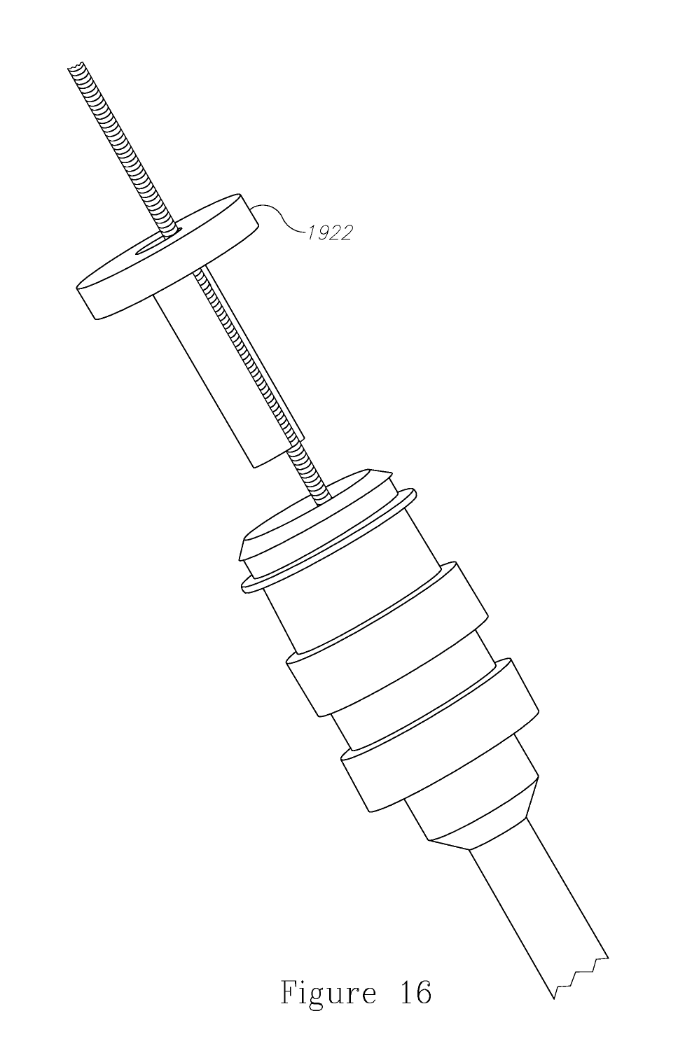

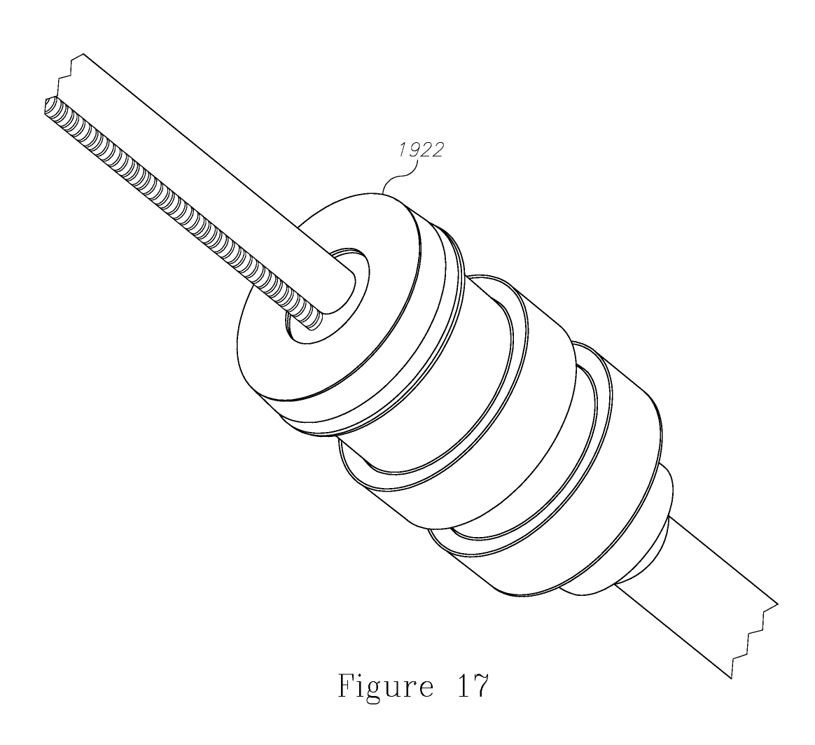

FIGS. 16 and 17 illustrate a silicon ring 1922. The ring 1922 serves to seal the external channel 1920 of the offset introducer and also the robotic arm. This is more clearly shown in FIG. 17.

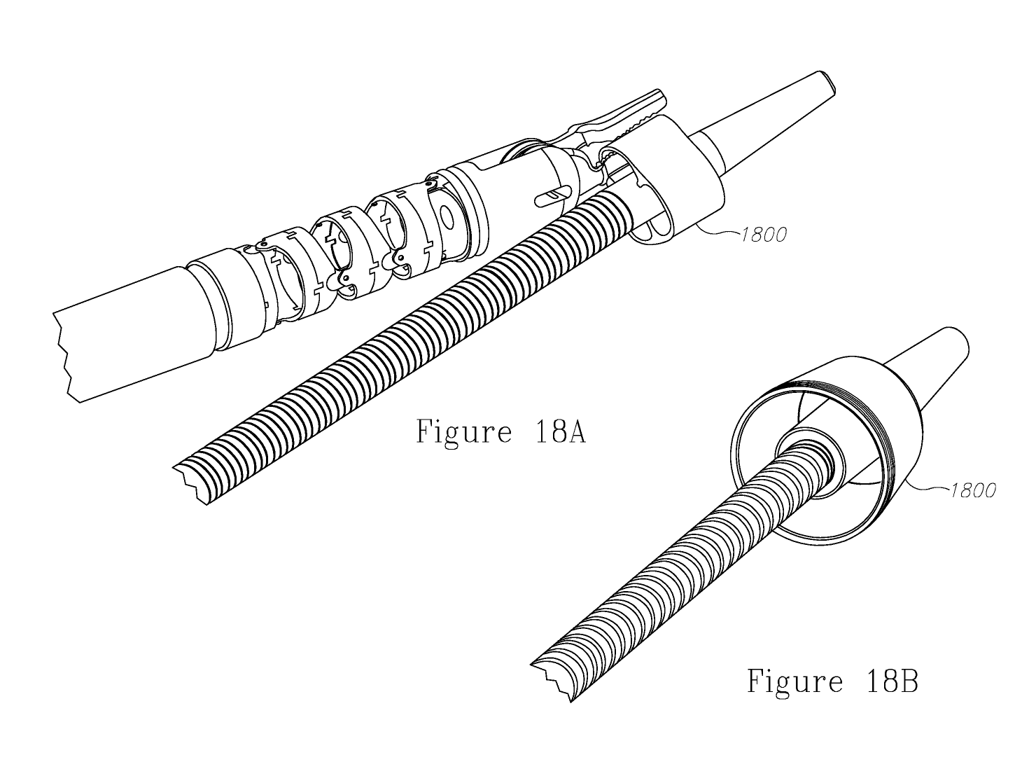

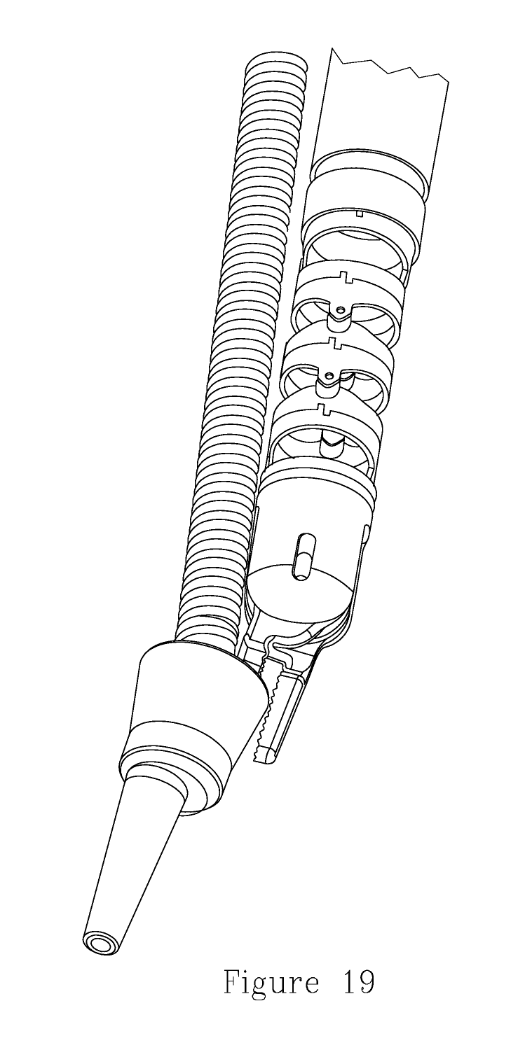

FIGS. 18a and 18b illustrate a collar 1800, via which the robotic arm grasps the waveguide tip. In FIG. 18a, the collar comprises several “petal” shapes via which the robotic arm is able to grasp the optical fiber waveguide tip. The “petal” shapes allow the robotic arm to firmly grasp the waveguide tip in a secure manner. In another embodiment, as shown in FIG. 18b, the collar is a continuous concentric ring—allowing the robotic arm to grasp the waveguide tip anywhere around the periphery of the collar. This is advantageous when the waveguide tip must be maneuvered in minute movements to allow the laser treatment to the patient's tissue. The collar allows the user to handle the optical fiber, and hence the laser beam, in a stable manner. This enables higher accuracy and higher resolution of the laser beam, and ensures the patient's safety.

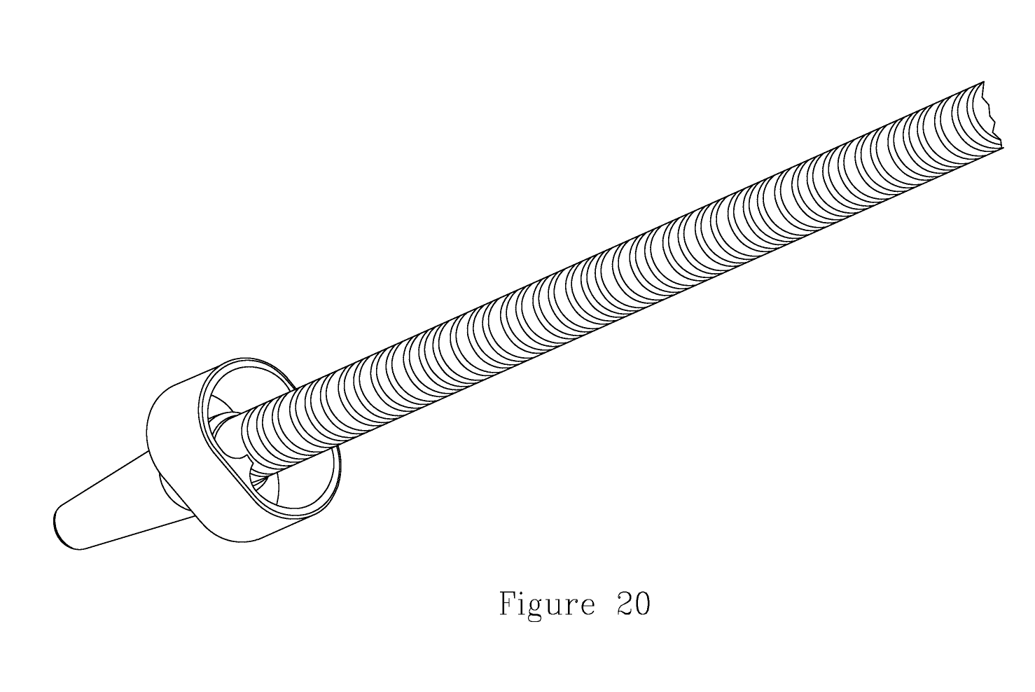

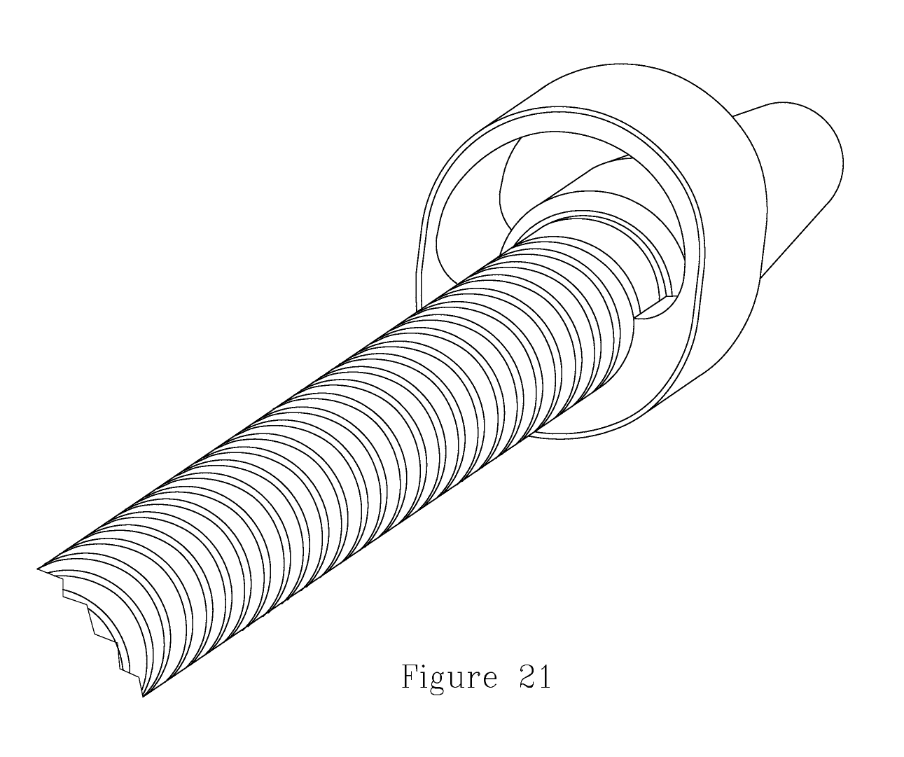

FIGS. 20 and 21 illustrate further embodiments of the collar provided around the end of the waveguide tip via which the robotic arm is able to grasp the laser instrument. In the embodiment shown in FIG. 20, the waveguide tip is symmetrical within the collar, in the embodiment shown in FIG. 21, the waveguide tip is non-symmetrical. This allows the robotic arm to maneuver the waveguide tip to a variety of positions necessary to treat the patient.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention. Therefore, it is manifestly intended that this invention be limited only by the claims and the equivalents thereof.

1. A system of surgical accessories for a surgical robotic arm, comprising:
at least one working channel coupled to an optical fiber;
a universal accessory adapter having:
a first end coupled to a working end of the surgical robotic arm,
a second end configured with an opening that exposes the at least one working channel; and
an accessory fastener,
wherein said universal accessory adapter is configured for securing an accessory device to the second end.

2. The system of claim 1, wherein the accessory fastener comprises either a threaded interface or a snap-on interface.

3. The system of claim 1, further comprising an accessory device having a fastening member configured to engage with the accessory fastener of the universal accessory adapter.

4. The system of claim 3, wherein the accessory device further comprises one of:
a waveguide tip accessory;
a bladed tip accessory;
a spatula accessory;
a backstop tip accessory; or
an offset fiber assembly.

5. The system of claim 1, wherein the accessory device further comprises a tip having a channel aligned with the working channel of the robotic arm, the tip further comprising a fiber locking feature.

6. The system of claim 5, wherein the fiber locking feature comprises a step down decrease in the diameter of the channel, towards the tip of the channel.

7. he system of claim 5, wherein the fiber locking feature comprises a continuous decrease in the diameter of the channel towards the tip of the channel.

8. The system of claim 1, wherein the universal accessory adapter further comprises a tip having a channel aligned with the working channel of the robotic arm, the tip further comprising a fiber locking feature.

9. The system of claim 6, further comprising an accessory device having a fastening member configured to engage with the accessory fastener of the universal accessory adapter.

10. The system of claim 7, wherein the accessory device further comprises one of:
a spatula accessory;
a backstop tip accessory; or
an offset fiber assembly.

11. The system of claim 1, further comprising:
at least one range restriction device coupled to the surgical robotic arm, wherein the at least one range restriction device is positioned around the surgical robotic arm in a location that at least partially restricts at least one degree of freedom of movement of the surgical robotic arm.

12. The system of claim 11, wherein the surgical robotic arm comprises a wrist assembly comprising a series of hinged links.

13. The system of claim 12, wherein the at least one range restriction device comprises at least one ring clamp attached to at least one of the hinged links, the at least one ring clamp having at least one axis limiter.

14. The system of claim 12, wherein the at least one range restriction device comprises a coiled spring assembly wrapped around the wrist assembly.

15. A surgical accessory for a surgical robotic arm having at least one working channel configured with an optical fiber, the accessory comprising:
an offset entry accessory comprising:
a ring member configured to attach to the surgical robotic arm; and
an offset fiber assembly coupled to the ring member,
wherein the offset entry accessory includes a first opening that delivers a first optical fiber from a working channel of the surgical robotic arm, and
wherein the offset fiber assembly includes a second opening that delivers a second optical fiber at an axis offset from the first optical fiber.

16. The accessory of claim 15, wherein the second optical fiber is coupled to a laser source.

17. The accessory of claim 15, wherein the second optical fiber is coupled to an imaging device.

18. The accessory of claim 15, wherein the offset entry accessory further comprises a tip having a channel offset from the working channel of the robotic arm, the tip further comprising a fiber locking feature.

19. The accessory of claim 15, further comprising:
at least one range restriction device coupled to the surgical robotic arm, wherein the at least one range restriction device is positioned around the surgical robotic arm in a location that at least partially restricts at least one degree of freedom of movement of the surgical robotic arm.

20. A surgical accessory, the accessory comprising:
a tip having a channel configured to deliver an optical fiber, the channel configured with a fiber locking feature that limits the fiber from penetrating through an opening of the tip;
a fiber guide tube holder that secures the optical fiber within the tip; and
a handle member configured for holding the tip.

21. The accessory of claim 20, wherein the handle member is a flipper.

22. The accessory of claim 20, wherein the handle member is a ring.

23. A surgical system, the system comprising:
a surgical robotic arm having a plurality of degrees of freedom of movement, and at least one working channel configured with an optical fiber;
a control station coupled to the surgical robotic arm, the control station configured to manipulate positioning of the robotic arm; and
a universal accessory adapter having a first end coupled to a working end of the surgical robotic arm, the universal accessory adapter having a second end configured with an opening that exposes the at least one working channel and an accessory fastener configured for securing an accessory device to the second end.

24. The system of claim 23, further comprising:
at least one range restriction device coupled to the surgical robotic arm, wherein the at least one range restriction device is positioned around the surgical robotic arm in a location that at least partially restricts at least one degree of freedom of movement of the surgical robotic arm.

25. The system of claim 24, wherein the surgical robotic arm comprises a wrist assembly comprising a series of hinged links.

26. The system of claim 25, wherein the at least one range restriction device comprises at least one ring clamp attached to at least one of the hinged links, the at least one ring clamp having at least one axis limiter.

27. The system of claim 25, wherein the at least one range restriction device comprises a coiled spring assembly wrapped around the wrist assembly.

28. The system of claim 23, wherein the accessory fastener comprises either a threaded interface or a snap-on interface.

29. The system of claim 23, further comprising an accessory device having a fastening member configured to engage with the accessory fastener of the universal accessory adapter.

30. The system of claim 23, further comprising:
a laser energy source coupled to the optical fiber; and
at least one secondary energy source coupled to the accessory device.

31. The system of claim 23, further comprising:
a laser energy source coupled to the optical fiber; and
an imaging device coupled to the accessory device.

32. The system of claim 31, wherein the accessory device further comprises one of:
a waveguide tip accessory;
a bladed tip accessory;
a spatula accessory;
a backstop tip accessory; or
an offset fiber assembly.

33. The system of claim 23, wherein the accessory device further comprises a tip having a channel aligned with the working channel of the robotic arm, the tip further comprising a fiber locking feature.

34. The system of claim 23, wherein the universal accessory adapter further comprises a tip having a channel aligned with the working channel of the robotic arm, the tip further comprising a fiber locking feature.

35. The system of claim 34, further comprising an accessory device having a fastening member configured to engage with the accessory fastener of the universal accessory adapter.

36. The system of claim 35, wherein the accessory device further comprises one of:
a spatula accessory;
a backstop tip accessory; or
an offset fiber assembly.
